(19) United States
(12) Patent Application Publication
(10) Pub. No.: US 2013/0338090 A1
(43) Pub. Date: Dec. 19, 2013

(54) Novel Compound Isolated from Allium tuberosum and Use Thereof as Antiviral Agent

(71) Applicants: Soo Ki Kim, Hanam-si (KR); Jun Hyeong Lee, Hanam-si (KR); Hyung In Moon, Seoul (KR); Sang Heui Seo, Daejeon (KR); Hyun Dong Paik, Seongnam-si (KR); Sang Buem Cho, Sangju-si (KR)

(72) Inventors: Soo Ki Kim, Hanam-si (KR); Jun Hyeong Lee, Hanam-si (KR); Hyung In Moon, Seoul (KR); Sang Heui Seo, Daejeon (KR); Hyun Dong Paik, Seongnam-si (KR); Sang Buem Cho, Sangju-si (KR)

(73) Assignees: Korea Health Industry Development Institute (KHIDI), Cheongwon-gun (KR); Konkuk University Industrial Cooperation Corp., Seoul (KR)

(21) Appl. No.: 14/002,140

(22) PCT Filed: Feb. 16, 2012
(86) PCT No.: PCT/KR12/01181

§ 371 Date: Aug. 29, 2013

(30) Foreign Application Priority Data
Mar. 3, 2011 (KR) 10-2011-0019044

(51) Int. Cl. C07H 17/07; C07H 1/08

(52) U.S. Cl. CPC C07H 17/07; C07H 1/08
USPC 514/27; 536/18.1

(57) ABSTRACT

The present invention relates to a novel compound isolated from Allium tuberosum and the use thereof as an antiviral agent. The compound isolated from Allium tuberosum shows the ability to inhibit highly pathogenic virus, and thus is a promising candidate for an antiviral agent.

### TECHNICAL FIELD

The present invention relates to a novel compound isolated from Allium tuberosum and the use thereof as an antiviral agent.

### BACKGROUND ART

The highly pathogenic avian influenza virus H5N1 which has recently emerged in Asia infects humans and shows a very high mortality (Yan, J. et al., 2007, China. Diagn. Microbiol. Infect. Dis. 58:399-405). In recent years, a new strain of influenza A virus subtype H1N1 has emerged and spread worldwide and was reported to have caused the deaths of about 4,000 persons (Ginocchio, C. C. et al., 2009, J. Clinical Virology 45: 191-195).

Also, this influenza virus attacks poultry, such as chickens or turkeys, and wild birds, and when it infects chickens and turkeys, it causes severe egg-production decline and economic damage due to a high communicability leading to a mortality of 100%. It is categorized as a type-1 legal communicable disease in Korea and list-A infectious disease by the Office International des Epizooties (OIE). Under such current circumstances, studies focused on finding avian influenza antiviral materials from natural plant extracts have been actively conducted (Mukhtar, M. et al., 2008, Virus Res. 131:111-120).

### DISCLOSURE OF INVENTION

### Technical Problem

The present invention has been made in view of the above-described need, and it is an object of the present invention to provide a novel antiviral substance extracted from a natural material.

Another object of the present invention is to provide a method of preparing a novel antiviral substance from a natural material.

Still another object of the present invention is to provide a method of preventing or treating viral disease by administering to a subject a novel antiviral substance extracted from a natural material.

Yet another object of the present invention is to provide the use of a novel antiviral substance, extracted from the natural substance, in preparation of a composition for preventing or treating viral disease.

### Solution to Problem

To achieve the above objects, the present invention provides a compound of the following formula 1:

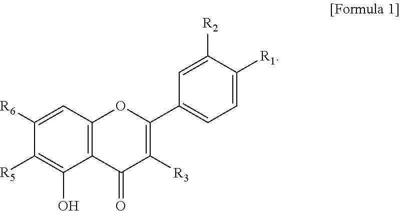

Preferably, the R₁ is OH, R₂ is H, R₃ is O-Rha-Glc or H, R₄ is H, and R₅ is OH or O-GlcUA, more preferably, the R₁ is OH, R₂ is H, R₃ is O-Rha-Glc, R₄ is H, and R₅ is OH.

The present invention also provides a method of obtaining the compound of the present invention from Allium tuberosum, the method comprising the steps of: treating Allium tuberosum with an organic solvent to obtain an organic solvent extract; suspending the organic solvent extract in water; treating the suspension with chloroform to obtain an chloroform fraction; and obtaining the compound of formula 1 from the chloroform fraction.

In one embodiment of the present invention, the organic solvent is preferably alcohol, for example, a C₁ to C₄ alcohol, in which the alcohol is preferably, but not limited to, methanol.

The present invention also provides a pharmaceutical composition for preventing or treating viral disease, the composition comprising the compound of the present invention or a pharmaceutically acceptable salt thereof.

The present invention also provides a feed composition for preventing or treating viral disease, the feed composition comprising the compound of the present invention or a chloroform fraction of Allium tuberosum.

In addition, the present invention provides the use of the compound of the present invention or a chloroform fraction of Allium tuberosum in preparation of a composition for preventing or treating viral disease.

In one embodiment of the present invention, the viral disease is preferably an influenza viral disease, in which the influenza virus preferably has a serotype selected from the group consisting of H5N1, H1N1, H1N2, H2N2, Human B, H3N2, H3N8, H5N2, H5N3, H5N8, H5N9, H7N1, H7N2, H7N3, H7N4, H7N7, H9N2 and H10N7, and more preferably the H5N1 serotype, but is not limited thereto.

As used herein, the term “Pharmaceutically acceptable salt” refers to any salt of the compound of this invention which retains its biological properties and which is not toxic or otherwise undesirable for pharmaceutical use. Such salts may be derived from a variety of organic and inorganic counterions well known in the art and include them. Such salts include: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxy-naphthoic, salicylic, stearic, cyclohexylsulfamic, quinic, muconic acid and the like acids; or (2) salts formed when an acidic proton present in the parent compound either (a) is replaced by a metal ion (e.g., an alkali metal ion, an alkaline earth ion or an aluminum ion), or alkali metal or alkaline earth metal hydroxides (e.g., sodium, potassium, calcium, magnesium, aluminum, lithium, zinc, and barium hydroxide), ammonia or (b) coordinates with an organic base, such as aliphatic, alicyclic, or aromatic organic amines, such as ammonia, methylamine, dimethylamine, di-ethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

In addition, examples of salts include sodium, potassium, calcium, magnesium, ammonium, tetraalkylammonium and the like, and when the compound contains a basic functionality, salts of non-toxic organic or inorganic acids, such as hydrohalides (e.g., hydrochloride and hydrobromide), sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate; fumarate, tartarate, citrate, benzoate, 3-(4-hydroxybenzoyl)benzoate, picrate, cinnamate, mandelate, phthalate, laurate, methanesulfonate (mesylate), ethane-sulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glu-coheptonate, 3-phenylpropionate, trimethylacetate, tert-butylacetate, lauryl sulfate, gluconate, benzoate, glutamate, hydroxynaphthoate, salicylate, stearate, cyclohexyl-sulfamate, quinate, muconate and the like.

In specific embodiments, the compound of the present invention may possess one or more asymmetric centers; this compound can therefore be produced as the individual (R)— or (S)-enantiomer or as a mixture thereof. Unless indicated otherwise, for example by designation of stereochemistry at any position of a formula, the description or naming of a particular compound in the specification and claims is intended to include both individual enantiomers and mixtures, racemic or otherwise, thereof. Methods for determination of stereochemistry and separation of stereoisomers are well-known in the art. In particular embodiments, the present invention provides the stereoisomer of the compound depicted herein upon treatment with base.

As used herein, the terms “subject” and “patient” are used interchangeably. The term “subject(s)” refers to an animal, preferably a mammal including a non-primate (e.g., a cow, pig, horse, cat, dog, rat, and mouse) and a primate (e.g., a monkey such as a cynomolgous monkey, a chimpanzee and a human), and more preferably a human. In one embodiment, the subject is refractory or non-responsive to current treatments for influenza infection. In another embodiment, the subject is a bird (e.g., a chicken, a turkey, a goose, a duck, a quail, a dove, or an ostrich), a farm animal (e.g., a horse, a cow, a pig, etc.) or a pet (e.g., a dog or a cat). In a preferred embodiment, the subject is a bird or human.

As used herein, the term “therapeutic agent(s)” refer to any agent(s) that can be used in the treatment, management, or amelioration of a disorder or one or more symptoms thereof. In certain embodiments, the term “therapeutic agent” refers to a compound of the present invention. In certain other embodiments, the term “therapeutic agent” does not refer to a compound of the present invention. In one embodiment, a therapeutic agent is an agent that is known to be useful for, or has been, or is currently being, used for the treatment or prevention of a disorder or one or more symptoms thereof.

“Therapeutically effective amount” means an amount of a compound or complex or composition that, when administered to a subject for treating a disease, is sufficient to effect such treatment for the disease. A “therapeutically effective amount” can vary depending on, inter alia, the compound, the disease and its severity, and the age, weight, etc., of the subject to be treated.

In one embodiment, “treating” or “treatment” of any disease or disorder refers to ameliorating a disease or disorder that exists in a subject. In another embodiment, “treating” or “treatment” refers to ameliorating at least one physical parameter that may be indiscernible by the subject. In yet another embodiment, “treating” or “treatment” refers to modulating the disease or disorder, either physically (e.g., stabilization of a discernible symptom) or physiologically (e.g., stabilization of a physical parameter) or both. In yet another embodiment, “treating” or “treatment” refers to delaying the onset of the disease or disorder.

As used herein, the terms “prophylactic agent(s)” refers to any agent(s) that can be used in the prevention of a disorder or one or more symptoms thereof. In certain embodiments, the term “prophylactic agent” refers to a compound of the invention. In certain other embodiments, the term “prophylactic agent” does not refer to a compound of the invention. Preferably, a prophylactic agent is an agent that is known to be useful for, or has been, or is currently being, used to prevent or impede the onset, development, progression and/or severity of a disorder.

As used herein, the terms “prevent,” “preventing,” and “prevention” refer to the prevention of the recurrence, onset, or development of one or more symptoms of a disorder in a subject resulting from the administration of a therapy (e.g., a prophylactic or therapeutic agent), or the administration of a combination of therapies (e.g., a combination of prophylactic or therapeutic agents).

As used herein, the phrase “prophylactically effective amount” refers to the amount of a therapy (e.g., prophylactic agent) that is sufficient to result in the prevention of the development, recurrence or onset of one or more symptoms associated with a disorder or to enhance or improve the prophylactic effect(s) of another therapy (e.g., another prophylactic agent).

The compound that is used in the method of the present invention is preferably provided using a pharmaceutical composition containing at least one compound of formula 1, if appropriate in the salt form, either used alone or in the form of a combination with one or more compatible and pharmaceutically acceptable carriers, such as diluents or adjuvants, or with another anti-HCV agent.

In clinical practice, the compound of the present invention may be administered by any conventional route, in particular orally, parenterally, rectally or by inhalation (e.g., in the form of aerosols).

As solid compositions for oral administration, tablets, pills, hard gelatin capsules, powders or granules may be used. In these compositions, the compound according to the present invention is mixed with one or more inert diluents or adjuvants, such as sucrose, lactose or starch. These compositions can comprise substances other than diluents, for example a lubricant, such as magnesium stearate, or a coating intended for controlled release.

As liquid compositions for oral administration, solutions which are pharmaceutically acceptable, suspensions, emulsions, syrups and elixirs containing inert diluents, such as water or liquid paraffin, may be used. These compositions may also comprise substances other than diluents, for example, wetting, sweetening or flavoring agents.

The compositions for parenteral administration may be emulsions or sterile solutions. As a solvent or a vehicle, propylene glycol, a polyethylene glycol, vegetable oils, in particular olive oil, or injectable organic esters, for example, ethyl oleate, may be used. These compositions may also contain adjuvants, in particular wetting, isotonizing, emulsifying, dispersing and stabilizing agents. Sterilization can be carried out in several ways, for example, using a bacteriological filter, by radiation or by heating. They can also be prepared in the form of sterile solid compositions that can be dissolved at the time of use in sterile water or any other injectable sterile medium.

The compositions for rectal administration are suppositories or rectal capsules that contain, in addition to the active ingredient, excipients such as cocoa butter, semi-synthetic glycerides or polyethylene glycols.

The compositions can also be aerosols. For use in the form of liquid aerosols, the compositions can be stable sterile solutions or solid compositions dissolved at the time of use in apyrogenic sterile water, in saline or any other pharmaceutically acceptable vehicle. For use in the form of dry aerosols intended to be directly inhaled, the active ingredient is finely divided and combined with a water-soluble solid diluent or vehicle, for example, dextran, mannitol or lactose.

In one embodiment, a composition of the present invention is a pharmaceutical composition or a single unit dosage form. Pharmaceutical compositions and single unit dosage forms of the present invention comprise a prophylactically or therapeutically effective amount of one or more prophylactic or therapeutic agents (e.g., a compound of the invention, or other prophylactic or therapeutic agent), and a typically one or more pharmaceutically acceptable carriers or excipients.

In a specific embodiment and in this context, the term “pharmaceutically acceptable” means approved by a regulatory agency of the Federal or a state government or listed in the Pharmacopeia or other generally recognized pharmacopeia for use in animals, and more particularly in birds or mammals, for example humans.

The term “carrier” refers to a diluent, adjuvant (e.g., Freund's adjuvant (complete and incomplete)), excipient, or vehicle with which the therapeutic agent is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously.

Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art, including the way in which the dosage form will be administered to a subject and the specific active ingredients in the dosage form. The composition or single unit dosage form, if desired, can also contain small amounts of wetting or emulsifying agents, or pH buffering agents.

The present invention further encompasses anhydrous pharmaceutical compositions and dosage forms comprising active ingredients. For example, the addition of water has been widely accepted in the pharmaceutical arts as a means of simulating long term storage in order to determine characteristics such as shelf life or the stability of formulations over time. See, e.g., Jens T. Carstensen, Drug Stability: Principles Practice, 2d. Ed., Marcel Dekker, NY, N.Y., 1995, pp. 379-80. In effect, water and heat accelerate the decomposition of some compounds. Thus, the effect of water on a formulation can be very important because moisture and/or humidity are commonly en-countered during manufacture, handling, packaging, storage, shipment, and use of formulations.

Anhydrous pharmaceutical compositions and dosage forms of the present invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected. An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The present invention also encompasses pharmaceutical compositions and dosage forms that comprise one or more compounds that reduce the decomposition rate of an active ingredient. Such compounds (referred to herein as “stabilizers”) include, but are not limited to, antioxidants (e.g., ascorbic acid), pH buffers, or salt buffers.

The pharmaceutical compositions and single unit dosage forms can take the form of solutions, suspensions, emulsion, tablets, pills, capsules, powders, sustained-release formulations and the like. A formulation for oral administration can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent preferably in purified form, together with a suitable amount of carrier, and provide the form for proper administration to the subject. The formulation should be suitable for the mode of administration. In a preferred embodiment, the pharmaceutical compositions or single unit dosage forms are sterile and in suitable form for administration to a subject, preferably an animal subject, more preferably an avian or mammalian subject, and most preferably a human subject.

A pharmaceutical composition of the present invention is formulated to be suitable for its intended route of administration. Examples of administration routes include, but are not limited to, parenteral, e.g., intravenous, intradermal, subcutaneous, intramuscular, subcutaneous, oral, buccal, sublingual, inhalation, intranasal, transdermal, topical, transmucosal, intra-tumoral, intra-synovial and rectal administration. In a specific embodiment, the composition is formulated in accordance with routine procedures as a pharmaceutical composition adapted for intravenous, subcutaneous, intramuscular, oral, intranasal or topical administration to humans. In one embodiment, a pharmaceutical composition is formulated in accordance with routine procedures for subcutaneous administration to humans.

Typically, compositions for intravenous administration are solutions in sterile isotonic aqueous buffer. If necessary, the composition may also include a solubilizing agent and a local anesthetic (e.g., lignocamne) to ease pain at the site of the injection.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a subject, including suspensions (e.g., aqueous or non aqueous liquid suspensions, oil-in-water emulsions, or a water-in-oil liquid emulsions), solutions, and elixirs; liquid dosage forms suitable for parenteral administration to a subject; and sterile solids (e.g., crystalline or amorphous solids) that can be reconstituted to provide liquid dosage forms suitable for parenteral administration to a subject.

The composition, shape, and type of dosage forms of the invention will typically vary depending on their use. For example, a dosage form used in the initial treatment of viral infection may contain larger amounts of one or more of the active ingredients it comprises than a dosage form used in the maintenance treatment of the same infection. Similarly, a parenteral dosage form may contain smaller amounts of one or more of the active ingredients it comprises than an oral dosage form used to treat the same disease or disorder. These and other ways in which specific dosage forms included in the present invention will vary from one another will be readily apparent to those skilled in the art. See, e.g., Remington's Pharmaceutical Sciences, 18th ed., Mack Publishing, Easton Pa. (1990).

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. When the composition is administered by infusion, it can be dispensed with an infusion bottle containing sterile pharmaceutical grade water or saline. When the composition is administered by injection, an ampoule of sterile water for injection or saline can be provided so that the ingredients may be mixed prior to administration.

Typical dosage forms of the present invention comprise a compound of the present invention, or a pharmaceutically acceptable salt, solvate or hydrate thereof in an amount ranging from about 0.1 mg to about 1000 mg/day, given as a single daily dose in the morning, but preferably as divided doses throughout the day taken with food. Particular dosage forms of the present invention have about 0.1, 0.2, 0.3, 0.4, 0.5, 1.0, 2.0, 2.5, 5.0, 10.0, 15.0, 20.0, 25.0, 50.0, 100, 200, 250, 500 or 1000 mg of the compound of formula 1.

Pharmaceutical compositions of the invention that are suitable for oral administration can be presented as discrete dosage forms, such as tablets (e.g., chewable tablets), caplet, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990). In specific embodiments, the oral dosage forms are solid and prepared under anhydrous conditions with anhydrous ingredients, as described in detail in the sections above.

However, the scope of the present invention extends beyond anhydrous, solid oral dosage forms. Typical oral dosage forms of the present invention are prepared by combining the active ingredient(s) in an intimate admixture with at least one excipient according to conventional pharmaceutical synthetic techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro crystalline cellulose, diluents, granulating agents, lubricants, binders, and disintegrating agents. Because of their ease of administration, tablets and capsules represent the most advantageous oral dosage unit forms when solid excipients are employed. If desired, tablets can be coated by standard aqueous or nonaqueous techniques. Such dosage forms can be prepared by any of pharmaceutical methods.

In general, pharmaceutical compositions and dosage forms are prepared by uniformly and intimately admixing the active ingredients with liquid carriers, finely divided solid carriers, or both, and then shaping the product into the desired presentation if necessary. For example, a tablet can be prepared by compression or molding. Compressed tablets can be prepared by compressing in a suitable machine the active ingredients in a free-flowing form (e.g., powder or granules), optionally mixed with an excipient. Molded tablets can be made by molding in a suitable machine a mixture of the powdered compound moistened with an inert liquid diluent.

Examples of excipients that can be used in oral dosage forms of the present invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed in the present invention include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler contained in pharmaceutical compositions of the present invention is typically present in an amount of about 50 to about 99 wt % based on the weight of the pharmaceutical composition or dosage form. Suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, Pa., USA), and mixtures thereof. A specific binder is a mixture of microcrystalline cellulose and sodium carboxymethyl cellulose sold as AVICEL RC 581. Suitable anhydrous or low moisture excipients or additives include AVICEL PH 103® and Starch 1500 LM.

Disintegrants are used in the compositions of the present invention to provide tablets that disintegrate when exposed to an aqueous environment. Tablets that contain too much disintegrant may disintegrate in storage, and those that contain too little may not disintegrate at a desired rate or under the desired conditions. Thus, a sufficient amount of disintegrant that is neither too much nor too little to detrimentally alter the release of the active ingredients should be used to form solid oral dosage forms of the present invention. The amount of disintegrant used varies based upon the type of formulation, and can be readily determined by those of ordinary skill in the art. Typical pharmaceutical compositions comprise about 0.5 to about 15 wt %, specifically about 1 to about 5 wt % of a disintegrant. Disintegrants that can be used in pharmaceutical compositions and dosage forms of the present invention include, but are not limited to, agar agar, alginic acid, calcium carbonate, microcrystalline cellulose, croscarmellose sodium, crospovidone, polacrilin potassium, sodium starch glycolate, potato or tapioca starch, pre gelatinized starch, other starches, clays, other algins, other celluloses, gums, and mixtures thereof.

Lubricants that may be used in pharmaceutical compositions and dosage forms of the present invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md., USA), a coagulated aerosol of synthetic silica (marketed by Degussa Co. of Plano, Tex., USA), CAB O SIL (a pyrogenic silicon dioxide product sold by Cabot Co. of Boston, Mass., USA), and mixtures thereof. If used, lubricants are typically used in an amount of less than about 1 wt % of the pharmaceutical compositions or dosage forms into which they are incorporated.

The compound of the present invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled release of one or more active ingredients using, for example, hydropropylmethyl cellulose, other polymer matrices, gels, permeable membranes, osmotic systems, multilayer coatings, microparticles, liposomes, microspheres, or combinations thereof to provide the desired release profile in varying proportions. Suitable controlled release formulations known to those of ordinary skill in the art, including those described in the present invention, can be readily selected for use with the active ingredient of the present invention. Thus, the present invention encompasses single unit dosage forms suitable for oral administration, for example, tablets, capsules, gelcaps, and caplets that are adapted for controlled release.

All controlled release pharmaceutical products have a common goal of improving drug therapy over that achieved by their non-controlled counterparts. Ideally, the use of an optimally designed controlled release preparation in medical treatment is characterized by a minimum of drug substance being employed to cure or control the condition in a minimum amount of time. Advantages of controlled release formulations include extended activity of the drug, reduced dosage frequency, and increased subject compliance. In addition, controlled release formulations can be used to affect the time of onset of action or other characteristics, such as blood levels of the drug, and can thus affect the occurrence of side (e.g., adverse) effects.

Most controlled release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions, including pH, temperature, enzymes, water, or other physiological conditions or compounds.

In specific embodiments, the agent may be administered using intravenous infusion, an implantable osmotic pump, a transdermal patch, liposomes, or other nodes of administration. In one embodiment, a pump may be used (see Sefton, CRC Crit. Ref. Biomed. Eng. 14:201 (1987)]; [Buchwald et al., Surgery 88:507 (1980)]; [Saudek et al., N. Engl. J. Med. 321:574 (1989)). In another embodiment, polymeric materials can be used. In yet another embodiment, a controlled release system can be placed in proximity of the therapeutic target, i.e., thus requiring only a fraction of the systemic dose. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

The present invention also provides parenteral dosage forms. Parenteral dosage forms can be administered to a subject by various routes, including subcutaneous, intravenous (including bolus injection), intramuscular, and intra-arterial. Because their administration typically bypasses patients' natural defenses against contaminants, parenteral dosage forms are preferably sterile or capable of being sterilized prior to administration to a subject. Examples of parenteral dosage forms include, but are not limited to, solutions ready for injection, anhydrous products that can be dissolved or suspended in a pharmaceutically acceptable vehicle for injection, suspensions for injection, and emulsions.

Suitable vehicles that can be used to provide parenteral dosage forms of the invention are known to those skilled in the art. Examples include, but are not limited to: water for injection (USP); aqueous vehicles such as sodium chloride injection, Ringer's injection, dextrose injection, dextrose and sodium chloride injection, and lactated ringer's injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate. Compounds that increase the solubility of one or more of the active ingredients disclosed herein can also be incorporated into the parenteral dosage forms of the present invention.

The present invention also provides transdermal, topical and mucosal dosage forms.

Transdermal, topical and mucosal dosage forms of the present invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th, 18th and 20th eds., Mack Publishing, Easton Pa. (1980, 1990 2000); and Introduction to Pharmaceutical dose form, 4th ed., Lea Febiger, Philadelphia (1985).

Dosage forms suitable for treating mucosal tissues within the oral cavity can be formulated as mouthwashes or as oral gels. Further, transdermal dosage forms include “reservoir type” or “matrix type” patches that can be applied to the skin and worn for a specific period of time to permit the penetration of a desired amount of active ingredients.

Suitable excipients (e.g., carriers and diluents) and other materials that can be used to provide transdermal, topical and mucosal dosage forms encompassed by the present invention are well known to those skilled in the pharmaceutical arts, and depend on the particular tissue to which a given pharmaceutical composition or dosage form will be applied. With that fact in mind, typical excipients include water, acetone, ethanol, ethylene glycol, propylene glycol, butane-1,3-diol, isopropyl myristate, isopropyl palmitate, mineral oil, and mixtures thereof, which form lotions, tinctures, creams, emulsions, gels or ointments that are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th, 18th and 20th eds., Mack Publishing, Easton Pa. (1980, 1990, 2000).

Depending on the specific tissue to be treated, additional components may be used prior to, in conjunction with, or subsequent to treatment with active ingredient of the present invention. For example, penetration enhancers can be used to assist in delivering the active ingredient to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts of the active ingredient can be used to further adjust the properties of the resulting composition.

In human therapeutics, the doctor will determine the posology which he considers most appropriate according to a preventive or curative treatment and according to the age, weight, stage of the infection and other factors specific to the subject to be treated. Generally, doses are from about 1 to about 1000 mg/day for an adult, or from about 5 to about 250 mg/day or from about 10 to 50 mg per/day for an adult. In certain embodiments, doses range from about 5 to about 400 mg/day per adult, and preferably 25 to 200 mg/day per adult. In a preferred embodiment, dose rates range from about 50 to about 500 mg/day.

In further aspects, the present invention provides methods of treating or preventing influenza viral infection in a subject in need thereof by administering to the subject an effective amount of a compound of the present invention, or a pharmaceutically acceptable salt thereof, with a high therapeutic index against influenza virus.

The therapeutic index can be measured according to any method known to those of skill in the art, such as the method described in the examples below. In certain embodiments, the therapeutic index is the ratio of a concentration at which the compound is toxic, to the concentration that is effective against influenza virus. Toxicity can be measured by any technique known to those of skill including cytotoxicity (e.g. IC₅₀ or IC₉₀) and lethal dose (e.g. LD₅₀ or LD₉₀). Likewise, effective concentrations can be measured by any technique known to those of skill including effective concentration (e.g. EC₅₀ or EC₉₀) and effective dose (e.g. ED₅₀ or ED₉₀). Preferably, similar measurements are compared in the ratio (e.g. IC₅₀/EC₅₀, IC₉₀/EC₉₀, LD₅₀/ED₅₀ or LD₉₀/ED₉₀). In certain embodiments, the therapeutic index can be as high as 2.0, 5.0, 10.0, 15.0, 20.0, 25.0, 50.0, 75.0, 100.0, 125.0, 150.0 or higher.

The amount of the compound or composition of the present invention that will be effective in the prevention, treatment, management or amelioration of a disorder or one or more symptoms thereof will vary according to the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

Exemplary doses of a composition include milligram or microgram amounts of the active compound per kilogram of subject or sample weight (e.g., about 10 μg/kg to about 50 μg/kg, about 100 μg/kg to about 25 mg/kg, or about 100 μg/kg to about 10 mg/kg).

For compositions of the present invention, the dosage administered to a subject is typically in the range of 0.140 mg/kg to 3 mg/kg of the subject's body weight, based on weight of the active compound. Preferably, the dosage administered to a subject is between 0.20 mg/kg and 2.00 mg/kg, or between 0.30 mg/kg and 1.50 mg/kg of the subject's body weight.

In general, the recommended daily dose range of a composition of the invention for the conditions described herein ranges from about 0.1 to about 1000 mg/day, given as a single once-a-day dose or as divided doses throughout a day. In one embodiment, the daily dose is administered twice daily in equally divided doses. Specifically, a daily dose range should be from about 10 to about 200 mg/day, more specifically, between about 10 and about 150 mg/day, or even more specifically between about 25 and about 100 mg/day. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition of the present invention are also encompassed by the above described dosage amounts and dose frequency schedules. Further, when a subject is administered multiple dosages of a composition of the invention, not all of the dosages need be the same. For example, the dosage administered to the subject may be increased to improve the prophylactic or therapeutic effect of the composition or it may be decreased to reduce one or more side effects that a particular subject is experiencing.

The present invention also provides a feed composition for preventing or treating viral disease, the feed composition comprising the compound of the present invention or a methanol fraction of Allium tuberosum.

To prepare the feed composition of the present invention, the Allium tuberosum fraction or compound of the present invention may be formulated alone or together with materials for feeds, such as wheat flour, starch, diluents such as dextrin, grain, bran such as chaff and defatted rice bran, and seed cakes having high oil and fat contents.

The feed composition of the present invention can be prepared in the form of a dried or liquid formulation and can additionally include one or more enzyme preparations. The additional enzyme preparation can also be in the form of a dried or liquid formulation and can be selected from a group consisting of lipolytic enzymes like lipase and glucose-producing enzymes such as amylase hydrolyzing the α-1,4-glycoside bond of starch and glycogen, phosphatase hydrolyzing organic phosphoric acid ester, carboxymethylcellulase decomposing cellulose, xylanase decomposing xylose, maltase hydrolyzing maltose into two glucose molecules, and invertase hydrolyzing saccharose into a glucose-fructose mixture.

In addition, the composition of the present invention may further comprise other non-pathogenic microorganisms. Examples of microorganisms that may be added to the composition of the present invention include, but are not limited to, Lactobacillus sp. strain having an ability to decompose organic compounds and physiological activity under anaerobic conditions (e.g., bovine stomach), filamentous fungi like Aspergillus oryzae (Slyter, L. L., J. Animal Sci. 1976, 43. 910-926) that increases the weight of domestic animals, enhances milk production and helps digestion and absorptiveness of feeds, and yeast like Saccharomyces cerevisiae (Johnson, D. E et al. J. Anim. Sci., 1983, 56, 735-739; Williams, P. E. V. et al, 1990, 211).

In addition, suitable feed raw materials such as crops, soybean protein, peanut, green pea, sugar beet, pulp, crop byproduct, animal intestine powder and fish powder can be used as they are or after being processed. To process the animal feed, a raw material for feed is compressed by pressure to be discharged. In the case of using a protein as a raw material, extrusion is preferably used in which the protein is denatured to increase its utilization. Extrusion has advantages in that it denatures a protein by heat-treatment, destructs anti-enzyme factors, and increases the activity of protease by enzymatic exposure to new sites resulting from molecular structural changes. In addition, in the case of using a soybean protein, extrusion can improve the digestibility of the protein, inactivate anti-nutrition factors such as trypsin inhibitor, one of the protease inhibitors, and increase digestibility by a protease, resulting in an increase in the nutritional value of the protein.

Typical examples of animals to which the composition of the present invention can be applied include, but are not limited to, birds, such as chickens, egg-producing hens, cocks, turkeys, geese, ducks, pheasants, quails, doves and ostriches, and domestic animals, such as pigs, piglets, beef cattle, milking cows, calves, sheep, goats, horses, rabbits, dogs and cats.

The dose of the feed composition will vary depending on the kind of animal to be administered with the composition, the age, and the kind of other feed components, and thus it is difficult to determine a constant dose. In general, the feed composition is preferably used as a feed additive that is added to formula feed in an amount of 0.01-10 wt % based on the weight of the formula feed. The feed composition of the present invention is particularly useful for the amelioration of clinical symptoms induced by avian influenza virus infection.

### Advantageous Effects of Invention

As disclosed in the present invention, the compound of the present invention, isolated from Allium tuberosum, shows the ability to inhibit highly pathogenic viruses, suggesting that it is a promising candidate for an antiviral agent.

### BEST MODE FOR CARRYING OUT THE INVENTION

Hereinafter, the present invention will be described in further detail with reference to non-limiting examples. It is to be understood, however, that these examples are for illustrative purposes only and are not intended to limit the scope of the present invention.

### Example 1

### Isolation and Purification of Active Ingredients from Extract Fractions of Allium tuberosum

In order to identify the ingredients of a chloroform fraction of Allium tuberosum, which showed the ability to inhibit highly pathogenic virus, 300 g of Allium tuberosum powder was prepared and used. Specifically, Allium tuberosum was dried in a dryer (Eyela, wfo-600SD) 65° C. for 2-3 days, and then powdered using a grinder (505, Daesung Artron Co., Ltd, Korea).

Extraction and fractionation were performed using EP solvent, column chromatography was performed using redistilled EP solvent, and TLC and HPLC were performed using HPLC solvent (Table 1 and Table 2).

TABLE 1

Column packing materials
Kiesel gel 60 (70-230 mesh, Merck, Art. 7734)
Kiesel gel 60 (less than 230 mesh, Merck, Art. 9385)
Sephadex LH-20 (bead size: 25-100 μm, Pharmacia)
Lichroprep RP-18 (bead size: 40-63 μm, Merck)
Lichroprep Si-60 (bead size: 40-63 μm, Merck Size A)
Lichroprep Si-60 (bead size: 40-63 μm, Merck Size B)
Thin layer chromatography
Kiesel gel60F₂₅₄ (precoatedplate, Art. 7552, Merck)
Spray material
Iodine vapor
10% H₂SO₄(inEtOH) sprayreagent
5% Phosphomolybdic acid (in EtOH) spray reagent

Table 1 above shows the materials used in the present invention.

TABLE 2

| Items | Laboratory instruments |
|---|---|
| mp | Gallenkamp melting point apparatus(uncorrected) |
| UV | Shimadzu UV spectrophotometer |
| FT-IR | JASCO FT/IR-5300 |
| ¹H-NMR | Bruker DPX-300 spectrometer (300 MHz) |
| ¹H-NMR | Bruker DMX-600 spectrometer (600 MHz) |
| ¹³C-NMR | Bruker DPX-300 spectrometer (75 MHz) |
| ¹³C-NMR | Bruker DMX-600 spectrometer (150 MHz) |
| HREI-MS | JEOL JMS-SX102WA (high resolution, 70 ev) |
| HRFAB-MS | JEOL HX-110WA (double focus MS, |
| | E/B Configuration) |
| Polarimeter | AUTOPOLIII automatic polarimeter |
| Recyclingpreparative | Recycling preparative HPLC LC-908 system |
| HPLC | (JAI co.) |
| Column | JAIGEL - 1H, pump: JAL pump |
| Detector | UV detector 310, RI detector RI-5 |
| Recycle | Autorecycle JAR-2 |
| Recorder | JAL SS-250F-2 |
| Semi-preparative | Gilson 850 system |
| HPLC | |
| Column | JAIGEL - ODS S-343-01 |
| Pump | Gilson 306 pump |
| Detector | Gilson UV detector 112 Shodex RI-71 |

Table 2 above shows the instruments used in the present invention.

300 g of Allium tuberosum was powdered, added to MeOH and extracted three times at room temperature during 2 weeks, followed by concentration in reduced pressure, and the resulting MeOH extract was suspended in water, and the suspension was treated with CHCl₃ and shaking extracted, and then the CHCl₃ layer is isolated using separatory funnel, followed by concentration in reduced pressure, thereby obtaining 6.3 g of a CHCl₃ fraction. 6.3 g of the CHCl₃ fraction of Allium tuberosum was subjected to C18 column chromatography, thereby obtaining the following fractions: H₂O, H₂O:CH₃CN (9:1→1:9), CH₃CN, CH₃CN:CH₃Cl(3:1), CH₃Cl, EtOH. Among these sub-fractions, the CH₃CN:CH₃Cl(3:1) fraction (89 mg) was subjected to silica gel column chromatography [developing solvent; EtOAc:n-hexane=2:10, 10:20, 20:50, 50:90, 0:100→EtOH/CH₃CN], thereby obtaining the fractions (79 mg) of n-hexane(20:50). And then compound I and compound II was isolated from the fraction preparative HPLC, which was named “Allium tuberosum compound I and II”.

### Example 2

### Identification of Structures of Allium tuberosum Compounds

2-1: Structure of Allium tuberosum Compound I

Allium tuberosum compound I, a powder of dark yellow color, showed positive reaction for FeCl₃. The negative FAB-MS spectrum showed a molecular ion peak of M-H— at m/z 593, a fragmention peak eliminated rhamnose at m/z 447, and a fragmention peak eliminated glucose and rhamnose at m/z 285.

The 1H-NMR spectrum showed doublet signal of J=8.75 Hz at δ7.98 ppm, the doublet signal was H-2′ and H-6′, and showed doublet signal of J=8.75 Hz at δ6.88 ppm, the doublet signal was H-5′ and H-3′. A signal of H-8 and 6 was a doublet by meta coupling at δ6.40 ppm and δ6.19 ppm, respectively. Anomeric proton of glucose (J=7.5 Hz) was detected at δ5.31 ppm, anomeric proton of rhamnose (J=3.4 Hz) was detected at δ4.37 ppm, and proton signal by CH₃ of rhamnose was detected at δ0.98 ppm. Based on the above results, this structure compared with the spectra described by Harbone J. and Marbry, T. 1993, The Flavonoids: Advance in Research., Campman and Hall. As a result, Allium tuberosum compound I was determined to have a structure of kaempferol 3-O-α-L-rhamnopyranosyl (1→6)-β-D-glucopyranoside as represented by the following formula 2:

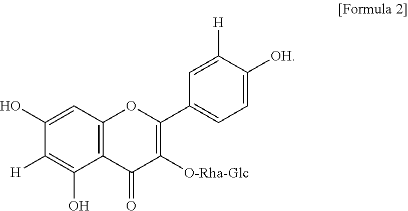

2-2: Structure of Allium tuberosum Compound II

Allium tuberosum Compound II, a powder of dark yellow color, showed positive reaction for FeCl₃. The negative FAB-MS spectrum showed a molecular ion peak of M-H— at m/z 445, a fragment ion peak eliminated glucuronic acid at m/z 269.

The ¹H-NMR spectrum showed doublet signal of H-2′ and H-6′ at δ7.96 ppm (2H, d, J=8.6 Hz), and showed doublet signal of H-3′ and H-5′ at δ6.95 ppm (2H, d, J=8.6 Hz), the H-3 was detected as singlet at δ6.87 ppm. A signal of H-6 and 8 was a doublet by meta coupling at δ6.86 ppm (1H, d, J=2.0 Hz) and 66.47 ppm (1H, d, J=2.0 Hz). And anomeric proton signal of glucuronic acid was detected as doublet at δ5.27 ppm (J=7.1 Hz). Based on the above results, this structure compared with the spectra described by Harbone J. and Marbry, T. 1993, The Flavonoids: Advance in Research., Campman and Hall. As a result, Allium tuberosum compound II was determined to have a structure of apigenin 7-O-β-D-glucuronopyranoside, C₂₁H₁₈O₁₁ as represented by the following formula 3:

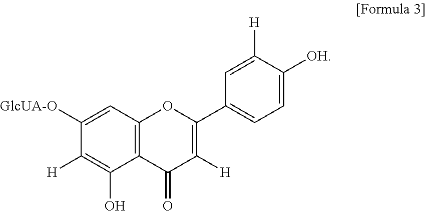

### Test Example 1

### Re-Verification of the Ability of Isolated Compounds to Inhibit Avian Influenza Virus

1-1: Preparation of Viral Strain

Testing for the verification of antiviral activity against highly pathogenic (H5N1) avian influenza was performed. The highly pathogenic virus used in the test was the A/Vietnam/1194/04(H5N1)-NIBRG-14 vaccine strain that was introduced after approval by the Korea Centers for Disease Control and Prevention, the World Health Organization (WHO) and the Centers for Disease Control and Prevention (CDC), and the test was performed in a biosafety level (BSA) 3+ facility, Chungnam National University.

1-2: Amplification of H5N1 Virus

H5N1 virus was inoculated into an 11-day-old embryonated egg and cultured in an incubator at 25° C. for 18 days, followed by cold storage in a refrigerator at 4° C. for 12 hours. Virus was collected from the allantoic cavity of the cold-stored embryonated egg and then stored in a freezer at −70° C. until use. Herein, the cultivation of H5N1 virus was performed in a 5% CO₂ incubator at 37° C. using a MEM (minimum essential medium) containing 10% (v/v) heat inactivated (37° C., 30 min) FBS (fetal bovine serum), penicillin and streptomycin.

1-3: Cell Cultivation

Host MDCK (Madin-Darby canine kidney) cells were cultured in a 5% CO₂ incubator at 37° C. using MEM (minimum essential medium) containing 10% (v/v) heat inactivated (37° C., 30 min) FBS (fetal bovine serum) and 1% penicillin and streptomycin, and then treated with 1× trypsin at 72-hr intervals, followed by splitting.

1-4: Measurement of Titer of Highly Pathogenic H5N1 Avian Influenza Virus

In order to examine the antiviral activity of a sample against H5N1, the H5N1 avian influenza virus that amplificated in the embryonated egg was 10-fold diluted in MEM medium and then inoculated into the cultured MDCK cells in a 96-well plate. After 48, the CPE (cytopathic effect) was observed to determine the tissue culture infectious dose (100TCID₅₀/), after which the viral titer was determined by a hemagglutination (HA) assay. When a group showed a decrease of 4 log units or more compared to a control, it was determined to have antiviral activity (Seo, S. H., et al., 2004, Virus Res. 103:107-11).

When the sample group showed a difference of 4 log units from the control group containing no sample, it was determined to be positive, and the control group containing no sample had a viral titer of 2⁷ HA units. When compared with the control group (2⁷ HA unit) containing no sample, the group containing compound I showed a viral titer of 2² HA units, which was 4-log lower than that of the control group. This suggests that the sample group had antiviral activity against highly pathogenic avian influenza H5N1, and the CPE inhibitory concentration was about 10 μg/ml (Table 3).

TABLE 3

| | | | CPE1 in- | | | |
|---|---|---|---|---|---|---|
| | | | hibitory | | | |
| | Solvent | | concentration | | | log100TCI |
| Testedplant | fraction | Compound | (μg/ml) | Activity | HA titer2 | D50/ml |
| Allium tuberosum | chloroform | I | 10 | ◯ | 2² | 5 |
| (Lee k) | | II | >10 | X | 2⁷ | 0 |

¹CPE cell pathogenic effect
²antiviral activity defined as a decrease of 4 log units

Table 3 above shows the antiviral activity of the compounds, derived from the chloroform fraction of Allium tuberosum, against avian influenza virus (H5N1).

1. A compound of the following formula 1: 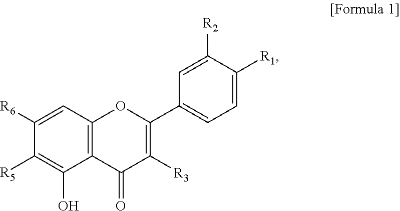
Wherein R₁ is OH, R₂ is H, R₃ is O-Rha-Glc or H, R₄ is H, and R₅ is OH or O-GlcUA.

2. A method of obtaining the compound of claim 1 from Allium tuberosum, the method comprising the steps of: treating Allium tuberosum with an organic solvent to obtain an organic solvent extract;
suspending the organic solvent extract in water; treating the suspension with chloroform to obtain an chloroform fraction; and obtaining the compound of claim 1 from the chloroform fraction.

3. The method of claim 2, wherein the organic solvent is a C₁ to C₄ alcohol.

4. The method of claim 3, wherein the alcohol is methanol.

5. A pharmaceutical composition for preventing or treating viral disease, comprising the compound of claim 1, or a pharmaceutically acceptable salt thereof.

6. The pharmaceutical composition of claim 5, wherein the viral disease is avian influenza viral disease.

7. The pharmaceutical composition of claim 6, wherein the avian influenza virus is H5N1-serotype avian influenza virus.

8. A feed composition for preventing or treating viral disease, comprising the compound of claim 1 or a methanol fraction of Allium tuberosum.

9. The feed composition of claim 8, wherein the viral disease is avian influenza virus.

10. The feed composition of claim 9, wherein the avian influenza virus is H5N1-serotype avian influenza virus.

11. A method for preventing or treating viral disease, comprising a step of administering an effective amount of the compound of claim 1 or a pharmaceutically acceptable salt thereof to a subject in need of prevention or treatment of viral disease.

12. The method of claim 11, wherein the viral disease is avian influenza viral disease.

13. The method of claim 12, wherein the avian influenza virus is H5N1-serotype avian influenza virus.

14. Use of the compound of claim or a methanol fraction of Allium tuberosum in preparation of a pharmaceutical composition for preventing or treating viral disease.
